(19) United States
(12) Patent Application Publication
(10) Pub. No.: US 2026/0036566 A1
(43) Pub. Date: Feb. 5, 2026

(54) DETERMINING HYDROCARBON MIGRATION DIRECTION USING AROMATIC-SELECTIVE SIZE EXCLUSION CHROMATOGRAPHY

(71) Applicant: Saudi Arabian Oil Company, Dhahran (SA)

(72) Inventors: Saroj Kumar Panda, Dhahran (SA); Khaled Arouri, Dhahran (SA); Tarik M. Al-Hoshan, Khobar (SA)

(21) Appl. No.: 18/790,646

(22) Filed: Jul. 31, 2024

(51) Int. Cl. G01N 33/28; G01N 30/02; G01N 30/86; G01N 30/88

(52) U.S. Cl. CPC G01N 33/2823; G01N 30/8693; G01N 30/88; G01N 33/2829; G01N 2030/027; G01N 2030/8854

(57) ABSTRACT

Alkylated aromatic compounds are present in all hydrocarbon samples. This technology uses aromatic selective size exclusion chromatography (ASSEC) to separate the alkylated aromatic compounds from the hydrocarbon samples. The hydrocarbon samples are obtained from various geographical locations in an oil basin. The distribution of the alkylated aromatic compounds is used to determine hydrocarbon migration indicators. A change in the distribution of the alkylated aromatic compounds between the hydrocarbon samples causes a change in the hydrocarbon migration indicators. The change in the hydrocarbon migration indicators between the hydrocarbon samples is used to determine the direction or pathway of hydrocarbon migration.

### TECHNICAL FIELD

This disclosure relates to hydrocarbon migration, and specifically to determining hydrocarbon migration direction or pathway across hydrocarbon fields.

### BACKGROUND

Hydrocarbon migration is the process by which hydrocarbons from a subterranean formation are expelled from a source rock. The hydrocarbons then flow into a permeable rock matrix. Several factors, such as kerogen expansion, pressure increase that pushes the hydrocarbon out of the source, gas expansion, thermal expansion, water motion due to compaction, and chemical potential, which relates to concentration differences, affect hydrocarbon migration. A precise determination of hydrocarbon generation, migration, and accumulation is a complex task that utilizes a range of analytical techniques to make the determination. Understanding the hydrocarbon migration pathways opens the possibility for determining a well drilling location accurately and avoiding dry holes.

### SUMMARY

This disclosure describes technologies relating to the determination of hydrocarbon migration indicators using aromatic-selective size exclusion chromatography (ASSEC). These hydrocarbon migration indicators are subsequently used to determine the direction of hydrocarbon migration. Having determined the direction in which the hydrocarbon has migrated, hydrocarbon extraction and production operations can be implemented in appropriate locations to which the hydrocarbon has migrated.

### BRIEF DESCRIPTION OF DRAWINGS

FIG. 1 is a schematic diagram of a system that uses ASSEC to separate several alkylated aromatic compounds from hydrocarbon samples.

FIG. 2 is a process flow diagram for the method of determination of hydrocarbon migration indicators using ASSEC.

FIG. 3 is a distribution plot of alkylated aromatic compounds from different crude oils collected from multiple migration pathways.

FIG. 4 is a tabulated data showing the hydrocarbon migration indicators for an oil basin using the ASSEC obtained distribution parameters.

FIG. 5 is an overlaid distribution plot of alkylated aromatic compounds from different crude oils.

FIG. 6 is a map showing the hydrocarbon migration pathways.

### DETAILED DESCRIPTION

Petroleum samples are complex mixtures. The factors that control the petroleum composition include the nature and thermal maturity of the generating source rock, migration from source to reservoir, pressure volume temperature (PVT) conditions, and alteration processes that the fluid will encounter within the reservoir. To study petroleum migration in a basin, hydrocarbon migration indicators, also known as geotracers, are used. Heterocycle compounds with nitrogen and sulfur are used as hydrocarbon migration indicators. Methods based on nitrogen- or sulfur-containing heterocycles are applicable only for crude oils that contain significant amounts of nitrogen or sulfur heterocycles. Crude oil samples such as gas condensates, especially those generated from overmatured source rocks, do not include significant amounts of nitrogen or sulfur.

Implementations described here use hydrocarbon migration indicators, which are based on the presence of alkylated aromatic compounds in a petroleum sample. The indicators based on the alkylated aromatic compounds are suitable for crude oils, gas condensates, and other classes of petroleum samples. The indicators based on the alkylated aromatic compounds can be applied equally in both conventional and unconventional petroleum systems, as it relies on the distribution of alkylated aromatic compounds that are inherently present in petroleum fluids of both systems. Additionally, the indicators based on the alkylated aromatic compounds can be applied in oil and gas condensate exploration, where known migration indicators (or geotracers), such as sulfur and nitrogen containing heterocyclic compounds are not sufficiently abundant.

Analytical techniques, such as comprehensive gas chromatography (GC) and high-resolution mass spectrometry (MS) are used in standalone or combined modes, for the characterization of aromatic species. The GC technique is not suitable for high-boiling fractions and residues, whereas the MS technique omits lighter compounds. The inherent limitations of both GC and MS techniques can compromise on the accuracy of measurement of aromatics in petroleum samples. Therefore, the aromatic-selective size exclusion chromatography (ASSEC) method is a suitable technique to analyze the complete range of petroleum samples, even those with low nitrogen or sulfur content.

In implementations described here, the application of the ASSEC technique can be extended to detect the alkylated aromatic compounds in petroleum samples. The ASSEC technique has a high detection limit. ASSEC takes a short time, e.g., in the order of minutes, for analysis and has a simple protocol. ASSEC involves the use of a high performance liquid chromatography (HPLC). The modern HPLC can be automated and does not involve a complex procedure.

In implementations here, hydrocarbon samples such as crude oils or gas condensates are obtained from various geographical locations in an oil basin. In some implementations, hydrocarbon samples are obtained from geographical locations that are spaced 5 kilometers (km) to 100 km from a source kitchen. These hydrocarbon samples include several alkylated aromatic compounds that are separated by ASSEC. Once the hydrocarbon sample (for example, crude oil) migrates from the source kitchen, the distribution of alkylated aromatic compounds changes. During crude oil migration, aromatic compounds with larger or longer alkyl carbon chains migrate preferentially over aromatic compounds with shorter alkyl chains. The distribution of several of these alkylated aromatic compounds is measured. The distribution is compared between the hydrocarbon samples obtained from various geographical locations to determine a change in the migration pathway or direction. Hydrocarbon migration pathway or direction in an oil basin is measured using the change in the distribution of the hydrocarbon migration indicators.

The start of the migration pathway is where the maturity of the source rock matches the maturity of the hydrocarbon sample. Source rock maturity is determined from basin modeling. The hydrocarbon sample maturity is determined from several maturity parameters, such as the vitrinite reflectance equivalence. Vitrinite reflectance equivalence can be calculated using the phenanthrene and methylphenanthrene distributions in the hydrocarbon sample using the methylphenanthrene index (MPI). Subsequently, hydrocarbon migration distance can be calculated as the distance between a source kitchen in the oil basin and a first field; and subsequently between each two successive fields measured on a map. The distance between fields is then simplified to be the map distance.

The ASSEC technique measures a retention time and an intensity for the alkylated aromatic compounds. An alkyl carbon atom number (Cp) and a weighted average alkyl carbon atom number (Cw) are determined from the retention time and the intensity of the alkylated aromatic compounds. An area below an alkyl carbon atom number N (AcN−), an area above the alkyl carbon atom number N (AcN+), and a ratio AcN−/AcN+ are calculated. The hydrocarbon migration direction or pathway is determined using the values of Cp, Cw, AcN−/AcN+ as indicators for hydrocarbon migration.

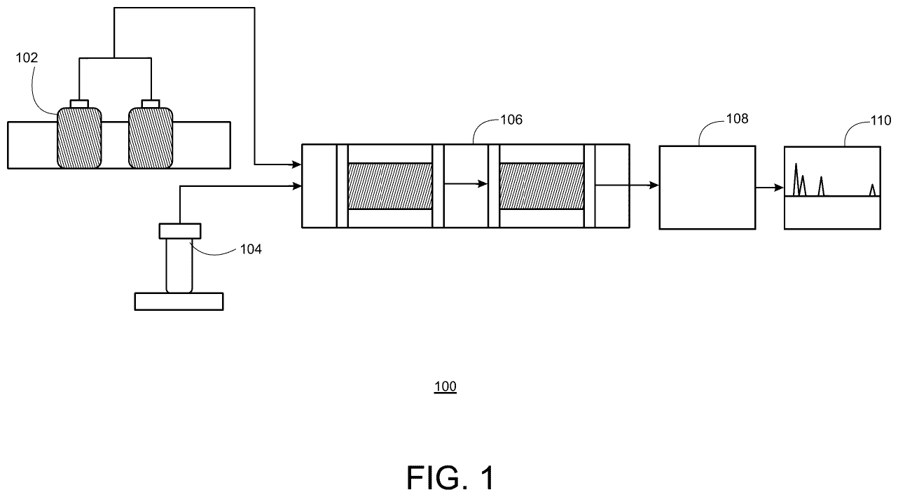

FIG. 1 is a schematic diagram of a system 100 that uses ASSEC to separate several alkylated aromatic compounds from hydrocarbon samples. ASSEC can be performed using a HPLC. The HPLC can include a binary pump through which the mobile phase 102 is delivered into the chromatographic column 106. A degasser can be used to remove air bubbles from the mobile phase 102. The mobile phase 102 used for the ASSEC method can include an organic solvent. In some implementations, the organic solvent can include toluene, dichloromethane, cyclohexane, or tetrahydrofuran.

The sample vial 104, which includes the hydrocarbon sample, is placed in an autosampler tray of the HPLC. In some implementations of the HPLC, a single chromatographic column can be used. In some implementations, two chromatographic columns 106 can be used. The chromatographic columns are placed in an oven for the measurements. The stationary phase used in the chromatographic columns 106 can include silica gel with a size range of 5 micrometers (μm)-100 Angstrom (Å) or a polymeric material. A diode array detector 108 can be used to record UV spectra from 200 nanometers (nm) to 400 nm.

Instrument control, data recording, and data analyses can be performed using a computer system that includes one or more processing devices and one or more machine-readable hardware storage devices. The machine-readable hardware storage device includes instructions that are executed by one or more processing devices to perform operations of the methods described in this disclosure.

In some implementations, the machine-readable instructions include a software that can be executed to perform the operations described here. Agilent Chemstation® is an example of such a software. The system 100 can include a display device 110, for example, connected to the computer system. The exemplary HPLC method parameters are presented in Table 1. An output of the HPLC method is a chromatogram, examples of which are described below.

TABLE 1
Parameters for ASSEC method

| | |
|---|---|
| Flow rate | 0.8 mL/min |
| Detector | DAD-UV detector (300 ± 100) nm |
| Injection volume | 2 μL |
| Run time | 30 minutes |
| Mobile phase | Dichloromethane |
| Column | Two HPLC columns (250 × 8 mm) packed with |
| | Eurospher II 100-5 NH2 connected in series |
| | (Average particle size and pore size of silica gel |
| | 5 μm and 100 Å, respectively) |
| Column oven | 30° C. |
| temperature | |

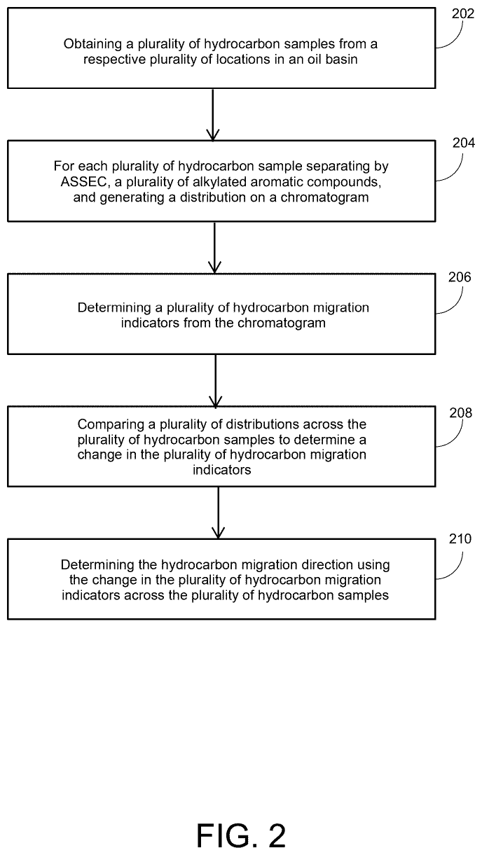

FIG. 2 is a process flow diagram for the method of determination of direction of hydrocarbon migration using ASSEC. At block 202, multiple hydrocarbon samples are obtained from various geographical locations within the same oil basin. The geographical locations have a spacing, ranging between 5-100 km. In some implementations, the geographical location can be close to the source kitchen in the oil basin.

At block 204, the ASSEC separation technique is applied to each of the multiple hydrocarbon samples. The hydrocarbon samples include light crude oil, medium crude oil, heavy crude oil, and gas condensates. The ASSEC is used to separate alkylated aromatic compounds in each hydrocarbon sample. The ASSEC is performed using the HPLC as described above. The HPLC uses a mobile phase that includes an organic solvent. In some implementations, the HPLC can include one column. In some implementations, the HPLC can include two columns. The columns can include a stationary phase. In some implementations, the stationary phase can include a silica gel or a polymeric material.

In response to the ASSEC separation technique, a chromatogram is obtained for each hydrocarbon sample. The chromatogram includes a distribution of alkylated aromatic compounds in each hydrocarbon sample. For example, the computer system described with reference to FIG. 1 is used to generate the chromatogram.

At block 206, for each hydrocarbon sample, several hydrocarbon migration indicators are determined from the chromatogram. The chromatogram includes the retention time and normalized intensity as the raw data. The raw data is fed into a prediction model (described later). The prediction model is used to determine the hydrocarbon migration indicators. The prediction model can be implemented by a computer system such as the one described with reference to FIG. 1. By repeating block 206 for each hydrocarbon sample, multiple distributions of alkylated aromatic compounds—one distribution per hydrocarbon sample—are determined.

At block 208, the distribution of the alkylated aromatic compounds is compared between the multiple hydrocarbon samples. The hydrocarbon samples are obtained from various geographical locations as described in block 202. By comparing the distributions of the alkylated aromatic compounds, as indicated on the multiple chromatograms generated for the respective multiple hydrocarbon samples, a change in the distribution across the various hydrocarbon samples is determined. Such comparing of the distributions can be implemented by a computer system such as the one described with reference to FIG. 1. To do so, the computer system can compare chromatograms via image analysis. Alternatively or in addition, the computer system can compare the tabulated values using which the chromatographs are generated. Using this change in the distribution, the computer system can determine a change in the hydrocarbon migration indicators, across the multiple hydrocarbon samples. In some implementations, the hydrocarbon migration indicators include Cp, Cw, the ratio AcN−/AcN+.

At block 210, the hydrocarbon migration direction in the oil basin can be determined using the change in the hydrocarbon migration indicators, across various hydrocarbon samples.

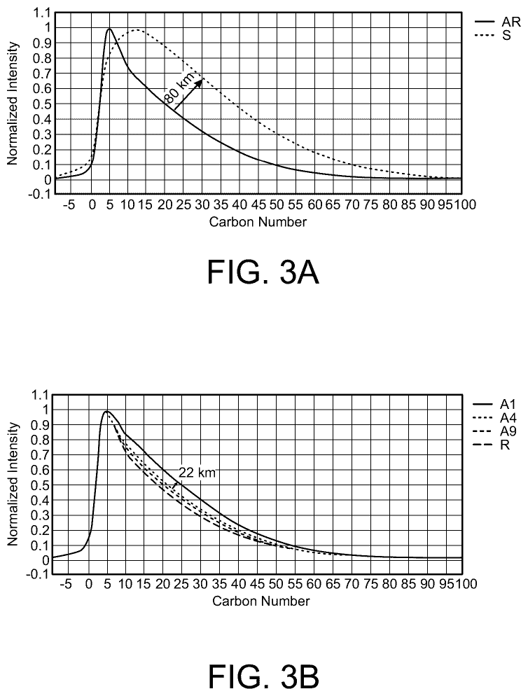

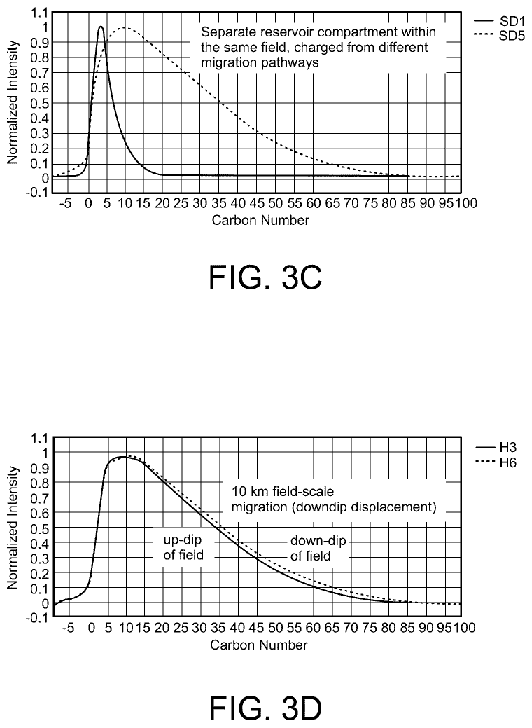

FIGS. 3A, 3B, 3C, and 3D are distribution plots of alkylated aromatic compounds from different crude oils collected from multiple migration pathways in a single basin. The distribution plot is obtained by performing ASSEC separation of a series of crude oils collected from a single oil basin. The crude oil samples are obtained from different geographic locations in the same oil basin. In some implementations, the crude oil samples are obtained from geographic locations that are spaced apart from each other, usually ranging between 5-100 km, although the distance between the fields could be less or more depending on the spacing of the traps where oil can accumulate. The distribution plots show the carbon number on the x-axis and the normalized intensity on the y-axis.

Crude oil samples include both aromatics and non-aromatic compounds. ASSEC technique involves the separation and analysis of aromatics, specifically the alkylated aromatics. The resulting raw data profile (retention time and intensity) of the chromatograms are exported and fed into a prediction model. The retention time data and a first prediction model are used to determine a physical parameter of the aromatic compound. A second prediction model determines the carbon number of an alkyl substituent of the aromatic compound using the physical parameter. For example, the physical parameter includes a molecular volume. A first prediction model includes a calibration curve of the molecular volume as a function of retention time. A second prediction model includes a calibration curve of the carbon number for the alkyl substituent as a function of molecular volume.

In some implementations, the aromatic compounds have a retention volume (Rᵥ) where:

$${R}_{v} = {R}_{t} x \qquad Eq . l$$

where x is the flow rate of the mobile phase used in the chromatography; Rₜ is the retention time of the aromatic compound.

In some implementations the physical parameter includes a molecular length. A first prediction model includes a calibration curve of the molecular length as a function of retention volume. A second prediction model includes a calibration curve of the carbon number for the alkyl substituent as a function of molecular length.

In some implementations, the first prediction model is constructed by measuring the retention times for standard aromatic compounds. A physical parameter is calculated for the standard aromatic compounds.

In some implementations, the carbon number for the alkyl substituent of the aromatic compounds includes an alkyl carbon number distribution, a highest alkyl carbon number, and/or an alkyl carbon number of highest abundance. In some implementations, the alkyl carbon number (also known as the alkyl carbon atom number) corresponding to the highest intensity located at the top of the peak of the carbon number vs intensity plot is indicated as Cp.

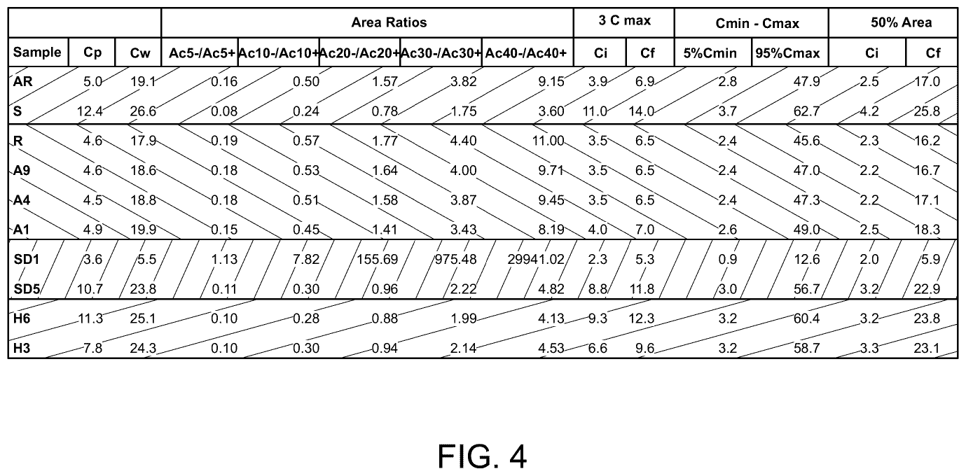

FIG. 4 is tabulated data showing the hydrocarbon migration indicators for an oil basin using the ASSEC obtained distribution parameters. In the present disclosure, the change in the distribution of alkylated aromatic compounds from various crude oil samples is analyzed. This change in the distribution of alkylated aromatic compounds is found to occur due to the migration of hydrocarbons. In some implementations, the following distribution parameters of alkylated aromatic compounds are used as hydrocarbon migration indicators.

Cp: Alkyl carbon atom number corresponding to the highest intensity located at the top of the peak. The value of Cp is calculated using the first prediction model and second prediction model as described in FIGS. 3A-D. Cw: Weighted average alkyl carbon atom number is calculated as:

$$\frac{{\int }_{{c}_{i}}^{{}^{c} f} c \cdot f ( c ) dc}{{c}_{f} - {c}_{i}} \qquad Eq . 2$$

where c represents the alkyl carbon atom number, f(c) represents the intensity at alkyl carbon atom number c. The integration spans from cᵢ to c_{f} (from the minimum carbon number to carbon number 100).

Ratio of AcN−/AcN+:area below alkyl carbon atom number N/area above alkyl carbon atom number N.

N C max (Ci and Cf): N carbon atom number interval showing the maximum intensity. Ci corresponds to initial carbon atom number and Cf corresponds to final carbon atom number.

Cmin: Cumulative abundance up to 5% (starting from lower carbon atom number).

Cmax: Cumulative abundance up to 95% (starting from lower carbon atom number).

P % Area (Ci and Cf): Narrowest interval covering P % area (Ci corresponds to initial carbon atom number and Cf corresponds to final carbon atom number).

All the above parameters are used to indicate hydrocarbon migration direction or pathway. Cp increases with increase in migration distance from the source kitchen.

Cw increases with increase in migration distance from the source kitchen.

Ratio of AcN−/AcN+ decreases with the increase in migration distance from the source kitchen.

Cmin and Cmax increase with the increase in migration distance from the source kitchen.

Ci and Cf increase with the increase in migration distance from the source kitchen.

In summary, as the hydrocarbon migration occurs in the subsurface the aromatic compounds with the longer alkyl chains preferentially move farther in comparison to the aromatic compounds with shorter alkyl chains. This causes different distribution patterns (or different ratios of short and long alkylated aromatic compounds) of alkylated aromatic compounds along the migration pathway. The increase or decrease in value of different hydrocarbon migration indicators helps in establishing hydrocarbon migration direction or pathway, as all the hydrocarbon migration indicators are derived from the distribution of alkylated aromatic compounds.

The ASSEC technique demonstrates that the aromatic compounds containing higher number of alkyl substituents (AcN+) preferentially migrate over lower number of alkyl substituents (AcN−) (Calculation of ratio AcN−/AcN+:Area below alkyl carbon atom number N/Area above alkyl carbon atom number N). N is the number of alkyl substituents in the aromatic compounds. N can range between 5-100. The ratio gradually decreases with the increase in hydrocarbon migration distance from the source, as aromatic compounds with higher alkyl carbon number preferentially migrate to farther distances. This trend is clear for long range migration, for example distance >20 km. Whereas the trend may not be that clear in short range migration, for example distance between 5-20 km. The increase or decrease in the value of the ratio AcN−/AcN+ helps in establishing hydrocarbon migration direction or pathway.

Depending on the hydrocarbon system (light oil, medium oil, heavy oil, or gas condensate), the distribution of alkylated aromatics will be different. In some implementations, this can extend to a higher number up to Ac100 and in other implementations, it can be up to Ac30. Therefore, various ranges such as Ac10−/Ac10+, Ac20−/Ac20+, Ac30−/Ac30+, Ac40−/Ac40+ or Ac100−/Ac100+ are chosen for the calculation of the ratio AcN−/AcN+.

The alkylated aromatic indicator Cp increases with the increase in hydrocarbon migration distance, as the aromatic compounds with the longer alkyl chains migrate farther in comparison with the aromatic compounds with the shorter alkyl chains. The indicator such as N C max (the interval of Ci and Cf), indicates that with an increase in Cf, there is an increase in hydrocarbon migration distance along the migration pathway. The increase or decrease in value of Cp or N C max helps in establishing hydrocarbon migration direction or pathway, as all the hydrocarbon migration indicators are derived from the distribution of alkylated aromatic compounds. The higher value of Cp or N C max indicates that the hydrocarbons have migrated farther away from the source kitchen along the migration pathway.

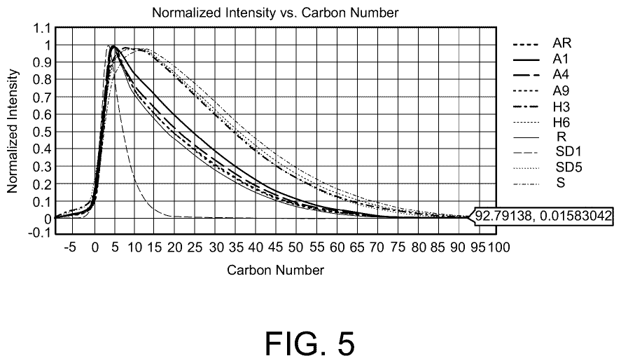

FIG. 5 is an overlaid distribution plot of alkylated aromatic compounds from different crude oil samples from the same oil basin. This figure shows the distribution of several alkylated aromatic compounds between different crude oil samples. The distribution parameters are obtained from the data in FIG. 4 and it is used to determine hydrocarbon migration pathways.

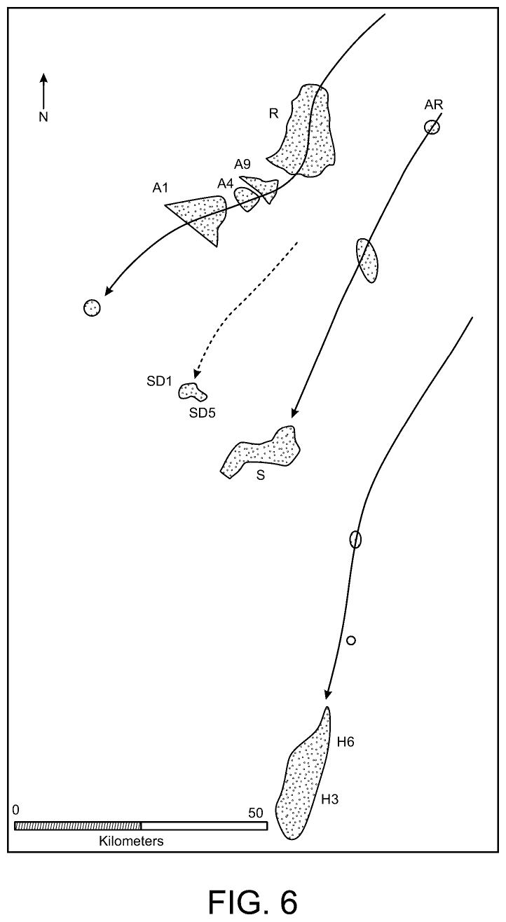

FIG. 6 is a map showing the hydrocarbon migration pathways. The map is a geographic surface map that shows the locations of various oil fields. Crude oil samples are taken from the top of the oil column in each field (which is usually the well drilled in the center of the field). The beginning of the migration pathway (where the oil maturity matches the source rock maturity) is determined to calculate the geographical distance travelled from the source kitchen to the first field. This is the geographical distance measured on the map. When the first field is filled, the oil is spilled up dip to the next trap (field), so on and so forth. This fill-spill sequence results in the systematic changes that are determined by the ASSEC technique.

The map demonstrates that there are four migration pathways for the hydrocarbons in the chosen oil basin. ASSEC indicates that the aromatic compounds containing higher number of alkyl substituents (AcN+) preferentially migrate over lower number of alkyl substituents (AcN−) (ratio of AcN−/AcN+:area below alkyl carbon atom number N/area above alkyl carbon atom number N). The ratio gradually decreases with the increase in migration distance as aromatic compounds with higher alkyl carbon number preferentially migrate to farther distance. The ratio also depends on the type of the hydrocarbon system, such as light oil, medium oil, heavy oil, or gas condensate.

Petroleum accumulated in multiple traps or fields are the result of either a single charge of single maturity or multiple charges of multiple maturities. In the case of single charge, no maturity interference is expected on the alkylated aromatic compounds. In the case of multiple charge, increased maturity will increase the concentration of the smaller alkyl substituents. This effect can improve the process of mapping migration, where the hydrocarbon fluids in the field closer to the source will be lighter, while fields farther away will have heavier hydrocarbon fluids.

An implementation described here relates to a method for the determination of hydrocarbon migration indicators. The hydrocarbon migration indicators are based on several alkylated aromatic compounds that are used to track hydrocarbon migration pathways (or direction) in both conventional and unconventional petroleum systems. Alkylated aromatic compounds can be applied to all types of crude oils including gas condensate because the principle of the application depends on ubiquitous aromatic compounds. This method can be applied to track hydrocarbon migration in complex basins that have large uncertainty regarding migration and where maturity variations are minimal. This method is applicable to light oils, medium oils, heavy oils, and gas condensates.

The implementations described here provide a method to locate hydrocarbon accumulations zones in a subterranean reservoir matrix, using the direction of hydrocarbon migration. The implementations described here provide a method to locate a drilling site based on the hydrocarbon accumulation in a subterranean reservoir matrix. The hydrocarbon migration indicators determine the direction or pathway of hydrocarbon migration, which is used for commercial oil and gas drilling operations. The methods in the implementations prevent drilling a dry hole.

### EXAMPLES

Certain aspects of the subject matter described here can be implemented as a method to determine hydrocarbon migration direction. Several hydrocarbon samples are obtained from respective locations in an oil basin. Each of these hydrocarbon samples include several alkylated aromatic compounds. For each of the hydrocarbon sample, the alkylated aromatic compounds are separated using an ASSEC technique. In response to the ASSEC technique, a chromatogram showing a distribution of several alkylated aromatic compounds is generated. Various hydrocarbon migration indicators are determined from the chromatogram. Distributions of the alkylated aromatic compounds are compared among several hydrocarbon samples to determine a change in the hydrocarbon migration indicators. The change in the hydrocarbon migration indicators, across the hydrocarbon samples, is used to determine the direction of hydrocarbon migration.

An aspect combinable with any other aspect includes the following features. For each of the hydrocarbon sample, various hydrocarbon migration indicators are used to determine the direction of hydrocarbon migration in the oil basin. The various hydrocarbon migration indicators include an alkyl carbon atom number (Cp) and a weighted average alkyl carbon atom number (Cw). Cw is based on Cp.

An aspect combinable with any other aspect includes the following features. Cp is determined from a prediction model.

An aspect combinable with any other aspect includes the following features. A retention time and a normalized intensity of the alkylated aromatic compounds from the chromatogram are used for the prediction model.

An aspect combinable with any other aspect includes the following features. The determination of one of the hydrocarbon migration indicators includes determining from the chromatogram, a first area below an alkyl carbon atom number N (AcN−) and a second area above the alkyl carbon atom number N (AcN+). A ratio of the first area and the second area (AcN−/AcN+) is determined.

An aspect combinable with any other aspect includes the following features. The alkyl carbon atom number N is chosen based on the hydrocarbon sample type. The hydrocarbon sample types include light oil, medium oil, heavy oil, or gas condensate.

An aspect combinable with any other aspect includes the following features. The alkyl carbon atom number N ranges between 5-100.

An aspect combinable with any other aspect includes the following features. The hydrocarbon samples are obtained from respective locations spaced apart between 5-100 km.

An aspect combinable with any other aspect includes the following features. The hydrocarbon samples include conventional and unconventional petroleum systems.

An aspect combinable with any other aspect includes the following features. The hydrocarbon samples include crude oil and gas condensates.

Certain aspects of the subject matter described here can be implemented as a method to determine hydrocarbon migration pathway. Hydrocarbon samples are obtained from two different locations of an oil basin, where the locations are spaced apart in a range of 5-100 km from a source in the oil basin. For each of the hydrocarbon sample, an ASSEC technique is used to separate the alkylated aromatic compounds. A retention time and an intensity for the alkylated aromatic compounds are measured to plot a distribution on a chromatogram, in response to the ASSEC technique. Various hydrocarbon migration indicators are determined from the chromatogram. The hydrocarbon migration indicators include an alkyl carbon atom number (Cp), a weighted average alkyl carbon atom number (Cw), and a ratio of a first area below an alkyl carbon atom number N (AcN−) and a second area above the alkyl carbon atom number N (AcN+). The hydrocarbon migration indicators between both the hydrocarbon samples are compared. A change is determined based on the comparison of the hydrocarbon migration indicators between both the hydrocarbon samples. The hydrocarbon migration pathway from the source in the oil basin is determined based on the change in the hydrocarbon migration indicators between both the hydrocarbon samples.

An aspect combinable with any other aspect includes the following features. The alkyl carbon atom number N ranges between 5-100.

An aspect combinable with any other aspect includes the following features. One or more machine-readable hardware storage devices that include instructions executable by one or more processing devices are used to determine the hydrocarbon migration indicators.

An aspect combinable with any other aspect includes the following features. The ASSEC technique includes an organic solvent as the mobile phase.

An aspect combinable with any other aspect includes the following features. The organic solvent includes toluene, dichloromethane, cyclohexane, or tetrahydrofuran.

An aspect combinable with any other aspect includes the following features. The ASSEC includes a stationary phase, where the stationary phase includes silica gel of particle size 5 μm.

An aspect combinable with any other aspect includes the following features. The hydrocarbon sample includes conventional or unconventional petroleum systems.

An aspect combinable with any other aspect includes the following features. The hydrocarbon sample includes light crude oil, medium crude oil, heavy crude oil, or gas condensates.

Certain aspects of the subject matter described here can be implemented as a system to perform a method to determine the direction of hydrocarbon migration. The system includes a HPLC to separate alkylated aromatic compounds from a hydrocarbon sample, using an ASSEC technique. A machine-readable hardware storage device that includes instructions executable by one or more processing devices is used to determine hydrocarbon migration indicators for the hydrocarbon sample.

An aspect combinable with any other aspect includes the following features. The HPLC includes a stationary phase which includes silica gel of particle size 5 μm and a mobile phase which includes an organic solvent.

Other implementations are also within the scope of the following claims.

1. A method to determine a direction of hydrocarbon migration, the method comprising:
obtaining a plurality of hydrocarbon samples from a respective plurality of locations in an oil basin, wherein each of the plurality of hydrocarbon samples comprises a plurality of alkylated aromatic compounds;
for each of the plurality of hydrocarbon samples:
separating, by an aromatic-selective size exclusion chromatography (ASSEC) technique, the plurality of alkylated aromatic compounds;
generating, in response to the ASSEC technique, a chromatogram showing a distribution of the plurality of alkylated aromatic compounds; and
determining a plurality of hydrocarbon migration indicators from the chromatogram;
comparing a plurality of distributions between the plurality of hydrocarbon samples to determine a change in the plurality of hydrocarbon migration indicators across the plurality of hydrocarbon samples; and
determining the direction of hydrocarbon migration in the oil basin using the change in the plurality of hydrocarbon migration indicators across the plurality of hydrocarbon samples.

2. The method of claim 1, wherein, for each of the plurality of hydrocarbon samples:
determining the direction of hydrocarbon migration in the oil basin comprises determining the plurality of hydrocarbon migration indicators wherein, the plurality of hydrocarbon migration indicators comprises:
an alkyl carbon atom number (Cp); and
a weighted average alkyl carbon atom number (Cw), based on Cp.

3. The method of claim 2, wherein Cp is determined from a prediction model.

4. The method of claim 3, wherein a retention time and a normalized intensity of the plurality of alkylated aromatic compounds from the chromatogram are used for the prediction model.

5. The method of claim 2, wherein, determining one of the plurality of hydrocarbon migration indicators comprises:
determining, from the chromatogram, a first area below an alkyl carbon atom number N (AcN−) and a second area above the alkyl carbon atom number N (AcN+); and
determining a ratio of the first area and the second area (AcN−/AcN+).

6. The method of claim 5, wherein, the alkyl carbon atom number N is chosen based on the plurality of hydrocarbon samples type, wherein the plurality of hydrocarbon samples type comprises light oil, medium oil, heavy oil, or gas condensate.

7. The method of claim 5, wherein the alkyl carbon atom number N ranges between 5-100.

8. The method of claim 1, wherein, the plurality of hydrocarbon samples is obtained from a respective plurality of locations spaced apart between 5-100 km.

9. The method of claim 1, wherein, the plurality of hydrocarbon samples comprises conventional and unconventional petroleum systems.

10. The method of claim 1, wherein the plurality of hydrocarbon samples comprises crude oil and gas condensates.

11. A hydrocarbon migration pathway determination method comprising:
obtaining a hydrocarbon sample from each of two different locations of an oil basin, wherein the locations are spaced apart in a range of 5-100 km from a source in the oil basin;
for each of the hydrocarbon sample:
separating, by an aromatic-selective size exclusion chromatography (ASSEC) technique, a plurality of alkylated aromatic compounds;
measuring a retention time and an intensity for the plurality of alkylated aromatic compounds to plot a distribution on a chromatogram, in response to the ASSEC technique;
determining a plurality of hydrocarbon migration indicators from the chromatogram, wherein, the plurality of hydrocarbon migration indicators comprise:
an alkyl carbon atom number (Cp);
a weighted average alkyl carbon atom number (Cw); and
a ratio of a first area below an alkyl carbon atom number N (AcN−) and a second area above the alkyl carbon atom number N (AcN+);
comparing the plurality of hydrocarbon migration indicators between both the hydrocarbon samples;
determining a change based on comparing the plurality of hydrocarbon migration indicators across both the hydrocarbon samples; and
determining the hydrocarbon migration pathway from the source in the oil basin, based on the change in the plurality of hydrocarbon migration indicators across both the hydrocarbon samples.

12. The method of claim 11, wherein the alkyl carbon atom number N ranges between 5-100.

13. The method of claim 11, wherein one or more machine-readable hardware storage devices comprising instructions that are executable by one or more processing devices are used to determine the plurality of hydrocarbon migration indicators.

14. The method of claim 11, wherein separating by the ASSEC comprises an organic solvent as a mobile phase.

15. The method of claim 14, wherein the organic solvent comprises toluene, dichloromethane, cyclohexane, or tetrahydrofuran.

16. The method of claim 11, wherein separating by the ASSEC comprises a stationary phase, wherein the stationary phase comprises silica gel of particle size 5 μm.

17. The method of claim 11, wherein the hydrocarbon sample comprises conventional or unconventional petroleum systems.

18. The method of claim 11, wherein the hydrocarbon sample comprises light crude oil, medium crude oil, heavy crude oil, or gas condensates.

19. A system to perform a method of claim 1, the system comprising:
a high performance liquid chromatography (HPLC) to separate a plurality of alkylated aromatic compounds from a hydrocarbon sample, using an aromatic-selective size exclusion chromatography (ASSEC) technique; and
a machine-readable hardware storage device comprising instructions that are executable by one or more processing devices to determine a plurality of hydrocarbon migration indicators for the hydrocarbon sample.

20. The system of claim 19, wherein the HPLC comprises:
a stationary phase which comprises silica gel of particle size 5 μm; and
a mobile phase which comprises an organic solvent.
